(19) United States
(12) Patent Application Publication
(10) Pub. No.: US 2008/0108633 A1
(43) Pub. Date: May 8, 2008

(54) Use 540

(75) Inventor: Alf Claesson, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/931,554

(22) Filed: Oct. 31, 2007

Related U.S. Application Data
(60) Provisional application No. 60/863,839, filed on Nov. 1, 2006.

(51) Int. Cl. A61K 31/505; A61P 25/00

(52) U.S. Cl. 514/256

(57) ABSTRACT

This invention relates to new use of pyrazolyl derivatives and pharmaceutically acceptable salts thereof, which have been found to possess analgesic activity and are accordingly useful in the treatment or prophylaxis of pain conditions in the human or animal body, for example in the manufacture of medicaments for the treatment or prevention of pain in a warm-blooded animal such as man.

### FIELD OF THE INVENTION

The invention concerns a new use of pyrazolyl derivatives and pharmaceutically acceptable salts thereof, which have been found to possess analgesic activity and are accordingly useful in the treatment or prophylaxis of pain conditions in the human or animal body, for example in the manufacture of medicaments for use in the treatment or prevention of pain in a warm-blooded animal such as man.

### BACKGROUND OF THE INVENTION

The current treatment regimes for pain conditions utilise compounds which exploit a very limited range of pharmacological mechanisms. One class of compounds, the opioids, stimulates the endogenous endorphine system; an example from this class is morphine. Compounds of the opioid class have several drawbacks that limit their use, e.g. emetic and constipatory effects and negative influence on respiratory capability. Their use is also restricted because of their addiction liabilities. The second major class of analgesics, the non-steroidal anti-inflammatory analgesics of the COX-1 or COX-2 types, also have liabilities such as insufficient efficacy in severe pain conditions and at long term use the COX-1 inhibitors cause ulcers of the mucosa. Mechanisms of analgesic effects of other currently used medicines are insufficiently characterized and/or have limited therapeutic potential.

Receptor tyrosine kinases (RTK's) are a sub-family of protein kinases that play a critical role in cell signalling and are involved in a variety of cancer related processes including cell proliferation, survival, angiogenesis and metastasis. Currently up to 100 different RTK's including tropomyosin-related kinases (Trk's) have been identified.

Trk's are the high affinity receptors activated by a group of soluble growth factors called neurotrophins (NT). The Trk receptor family has three members—TrkA, TrkB and TrkC. Among the NTs there are (i) nerve growth factor (NGF) which activates TrkA, (ii) brain-derived growth factor (BDNF) and NT-4/5 which activate TrkB and (iii) NT3 which activates TrkC. Each Trk receptor contains an extra-cellular domain (ligand binding), a trans-membrane region and an intra-cellular domain (including kinase domain). Upon binding of the ligand, the kinase catalyzes auto-phosphorylation and triggers downstream signal transduction pathways.

Trk's are widely expressed in neuronal tissue during its development where Trk's are critical for the maintenance and survival of these cells. A post-embryonic role for the Trk/neurotrophin axis (or pathway), however, remains in question. There are reports showing that Trk's play important role in both development and function of the nervous system (Patapoutian, A. et al Current Opinion in Neurobiology, 2001, 11, 272-280).

In the past decade, many scientific reports have been published which link Trk signaling with induction of pain. Levels of NGF are increased after inflammation and NGF contributes to basal and stimulus-induced hyperalgesia (for example, Safieh-Garabedianof et al. British Journal of Pharmacology 1995, 115, 1265). After inflammation BDNF levels are also increased in dorsal root ganglion as indicated by increased mRNA levels (Cho et al. Brain Reseach 1997, 749, 358). Strong support for the involvement of TrkA/TrkB and their ligands NGF/BDNF in pain comes from studies utilizing antibodies towards NGF or fusion proteins of Trk receptors with immunoglobulins which scavenge NGF or BDNF. Several such studies have shown analgesic effects in animals in which inflammation has been induced (for example, Lewin et al. European Journal of Neuroscience 1994, 6, 1903; McMahon et al. Nature Medicine 1995, 1, 774). Although the studies do not deal with the Trk receptor kinases per se they indicate that inhibition of the NGF or BDNF receptor coupled tyrosine kinase may also lead to analgesic effects.

Recent literature also indicates that activation of TrkA with NGF causes downstream upregulation of certain ion channels which are important in increasing the electric signaling from the nerve endings which experience the inflammation, thus inducing pain (for example, VR-1, Winston et al. Pain 2001, 89, 181; sodium channels, Choi et al. Molecular and Cellular Biology 2001, 21, 2695; ASIC, Mamet et al. Journal of Biological Chemistry 2003, 278, 48907).

NGF has also been implicated as a factor causing psoriasis and pruritis (itch) indicating that a TrkA inhibitor may be used for these indications (British Journal of Dermatology 2006, 155, 876).

We have now found surprisingly that certain pyrazolyl derivatives possess potent analgesic activity by acting as inhibitors of TrkA and TrkB.

There are few reports of selective Trk tyrosine kinase inhibitors that are highly selective for TrkA and TrkB. Cephalon described CEP-751, CEP-701 (George, D. et al Cancer Research, 1999, 59, 2395-2401) and other indolocarbazole analogs (WO0114380) as Trk inhibitors. It was shown that the alkaloid K252a, which is related to CEP-701/751, when injected into rats with pancreatite could suppress mechanical hypersensitivity (Winston et al. Journal of Pain 2003, 4, 329).

It is disclosed in patent application JP 2003-231687 that pyrazole compounds condensed with cycloalkylenes in the 4,5-positions act as neurotrophin receptor inhibitors and can be used as painkillers. GlaxoSmithKline disclosed certain oxindole compounds as TrkA inhibitors and as useful for the treatment of pain and cancer (WO0220479, WO0220513)

It is disclosed in patent applications WO0250065 and WO0262789 from Vertex Pharmaceuticals that pyrazole compounds are inhibitors of GSK3, Aurora, etc. and are useful for the treatment of cancer. AstraZeneca PLC reported pyrazole compounds as inhibitors of IGF-1 receptor kinase (WO0348133).

### DESCRIPTION OF THE FIGURES

FIG. 1. Hindpaw weight distribution at different doses.

### DESCRIPTION OF THE INVENTION

The invention relates to the use of compounds selected from
(S)-5-bromo-N²-(1-(5-fluoropyridin-2-yl)ethyl)-N⁴-(5-isopropoxy-1H-pyrazol-3-yl)pyrimidine-2,4-diamine;
5-chloro-N²-[(1S)-1-(5-fluoropyridin-2-yl)ethyl]-N⁴-(5-isopropoxy-1H-pyrazol-3-yl)pyrimidine-2,4-diamine;
(S)-5-bromo-N²-(1-(3,5-difluoropyridin-2-yl)ethyl)-N⁴-(5-isopropoxy-1H-pyrazol-3-yl)pyrimidine-2,4-diamine;
(S)-5-chloro-N²-(1-(3,5-difluoropyridin-2-yl)ethyl)-N⁴-(5-isopropoxy-1H-pyrazol-3-yl)pyrimidine-2,4-diamine;
5-fluoro-N²-[(1S)-1-(5-fluoropyridin-2-yl)ethyl]-N⁴-(5-isopropoxy-1H-pyrazol-3-yl)pyrimidine-2,4-diamine;
(S)—N²-(1-(3,5-difluoropyridin-2-yl)ethyl)-5-fluoro-N⁴-(5-isopropoxy-1H-pyrazol-3-yl)pyrimidine-2,4-diamine;
(S)-5-bromo-N⁴-(5-cyclopropyl-1H-pyrazol-3-yl)-N²-(1-(5-fluoropyridin-2-yl)ethyl)pyrimidine-2,4-diamine;
(S)-5-chloro-N⁴-(5-cyclopropyl-1H-pyrazol-3-yl)-N²-(1-(5-fluoropyridin-2-yl)ethyl)pyrimidine-2,4-diamine;
(S)-5-chloro-N⁴-(5-cyclopropyl-1H-pyrazol-3-yl)-N²-(1-(3,5-difluoropyridin-2-yl)ethyl)pyrimidine-2,4-diamine; and
5-chloro-N²-[(1S)-1-(5-fluoropyridin-2-yl)ethyl]-N⁴-(5-methyl-1H-pyrazol-3-yl)pyrimidine-2,4-diamine,
   or pharmaceutically acceptable salts thereof, in the manufacture of a medicament for treatment or prophylaxis of pain.

The compounds of the invention as mentioned above can be prepared according to processes described in WO2006/123113.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine. In a further aspect of the invention, a suitable pharmaceutically acceptable salt of a compound of the inventions, particularly a compound selected from any one of the Examples, is a salt formed with an acid selected from: benzoic acid, 2-(benzoylamino)acetic acid, 1,2-ethane disulfonic acid, fumaric acid, maleic acid, mandalic acid, naphthalene-1,5-disulfonic acid, phosphoric acid, succinic acid, sulfuric acid or undec-10-enoic acid. In one aspect the salt is a phosphate. In another embodiment the salt is a sulphate. In a further aspect the salt is a fumarate. In a further embodiment the salt is a maleate.

Further examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate.

Examples of base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

It is also to be understood that certain compounds of the invention can exist in solvated as well as unsolvated forms such as, for example, hydrated forms. It is to be understood that the invention encompasses all such solvated forms.

Usage

The compounds as mentioned above, or pharmaceutically acceptable salts thereof, which possess kinase inhibitory activity are contemplated to be useful in the treatment or prophylaxis of pain conditions and are therefore believed to be useful in methods of treatment of human or animal body. The invention also relates to the use of pharmaceutical compositions comprising said compounds and to their use in the manufacture of medicaments for the production of analgesic effect in warm-blooded animals such as man.

The present invention includes use of pharmaceutically acceptable salts or pro-drugs of such compounds.

The properties of the compounds used according to the claimed invention are expected to be useful in therapy, especially for the treatment and/or prophylaxis of pain which may be of widely different origins and causes and include acute as well as chronic pain states. Examples are pain caused by chemical, mechanical, radiation, thermal, infectious or inflammatory tissue trauma or cancer. Additional examples are posttraumatic pain, headache and migraine, various arthritic and inflammatory conditions such as osteo and rheumatoid arthritis, myofascial and low back pain associated with chronic inflammation, bone diseases, and cell proliferation such as cancers (solid tumors and leukemia).

Also neuropathic conditions of central or peripheral origin may be treated or prevented using compounds as mentioned above, or pharmaceutically acceptable salts thereof. Examples of these pain conditions are trigeminal neuralgia, postherpetic neuralgia (PHN), painful diabetic mono/poly neuropathy, and pain associated with nerve damage, spinal cord injury, central post stroke, multiple sclerosis, psoriasis, pruritis and Parkinson's disease.

Other pain may have a visceral origin such as pain caused by ulcer, dysmenorrhea, endometriosis, IBS, dyspepsia etc. and may also be treated or prevented with the compounds as mentioned above, or pharmaceutically acceptable salts thereof.

One embodiment of the invention relates to the use of a compound, as mentioned above, or a pharmaceutically acceptable salt thereof, in the manufacture of a medicament for treatment or prophylaxis of pain.

Another embodiment of the invention relates to the use of a compound, as mentioned above, or a pharmaceutically acceptable salt thereof, in the manufacture of a medicament for treatment or prophylaxis of inflammatory pain or neuropathic pain.

In a further embodiment the inflammatory pain is chronic inflammatory pain.

In another embodiment of the invention the use is therapeutic.

In yet another embodiment of the invention the use is prophylactic.

In one embodiment of the invention said pain is selected from pain caused by chemical, mechanical, radiation, thermal, infectious or inflammatory tissue trauma.

In a further embodiment of the invention said pain is posttraumatic pain, or pain caused by headache and migraine, arthritic and inflammatory conditions selected from pain caused by osteo arthritis and rheumatoid arthritis, myofascial and low back pain associated with chronic inflammation, bone diseases or cell proliferation such as cancers (solid tumors and leukemia).

In yet a further embodiment of the invention said pain is of central or peripheral origin, selected from pain caused by trigeminal neuralgia, postherpetic neuralgia (PHN), painful diabetic mono/poly neuropathy, and pain associated with nerve damage, spinal cord injury central post stroke, multiple sclerosis or Parkinson's disease.

In one embodiment of the invention said pain is of visceral origin selected from pain caused by ulcer, dysmenorrhea, endometriosis, IBS and dyspepsia.

Another embodiment of the invention relates to a method of treatment or prophylaxis of pain, (chronic) inflammatory pain or neuropathic pain, and any pain mentioned above, in a patient suffering from, or at risk of, said disease, which comprises administering to the patient a therapeutically effective amount of a compound, as mentioned above, or a pharmaceutically acceptable salt thereof.

A further embodiment relates to an agent for the treatment or prophylaxis of pain, chronic inflammatory pain or neuropathic pain, and any pain mentioned above, which comprises as active ingredient a compound, as mentioned above, or a pharmaceutically acceptable salt thereof.

The dosage will depend on the route of administration, the severity of the disease, age and weight of the patient, the disease of the patient, and other factors normally considered by the attending physician, when determining the individual regimen and dosage level as the most appropriate for a particular patient.

An effective amount of a compound used according to the present invention for use in therapy of pain is an amount sufficient to symptomatically relieve in a warm-blooded animal, particularly a human, the sensations of pain, to slow the progression of pain sensations, or to reduce in patients with pain the risk of experiencing worse pain.

In general, the dosages will be in the range of 1 to 1000 mg per day of active substance.

Another embodiment of the invention relates to the uses mentioned above, wherein the daily dose of the compounds as mentioned above is in the range of from about 0.1 mg to about 1000 mg.

A further embodiment of the invention relates to the uses mentioned above, wherein the daily dose of the compounds as mentioned above is in the range of from about 1 mg to about 750 mg.

One embodiment of the invention relates to the uses mentioned above, wherein the daily dose of the compounds as mentioned above is in the range of from about 1 mg to about 500 mg.

The wording “daily dose” is defined so that the pyrazolyl compounds may be given either as a unit dosage once daily, such as a tablet or a capsule, or alternatively the pyrazolyl compounds may be given twice daily. The daily dose may vary within the dosage ranges mentioned below, and depends on the patient's individual response to treatment.

With the wording “therapeutic treatment” as herein used, is meant that pain is treated by administering compounds of invention or a pharmaceutically acceptable salt thereof, as mentioned above, as soon as the pain has started to give the patient suffering therefrom, to relieve pain sensations. This means that the use of said compounds, provides therapy of a fully or partly developed pain condition such as pain caused by chemical, mechanical, radiation, thermal, infectious or inflammatory tissue trauma or cancer.

With the wording “prophylactic treatment” as herein used, is meant that a pyrazolyl derivative according to the invention, may be administered to a person to prevent the frequency of pain attacks and to reduce the severity or the duration of the attack. Furthermore, it may be administered before the pain attack has started to give full symptoms or only slight symptoms.

Pharmaceutical Composition

In order to use a compound of the invention or a pharmaceutically acceptable salt thereof for the therapeutic treatment (including prophylactic treatment) of mammals including humans, it is normally formulated in accordance with standard pharmaceutical practice as a pharmaceutical composition.

One embodiment of the invention relates to the use of a pharmaceutical composition comprising the compounds of the invention, as mentioned above, or pharmaceutically acceptable salts thereof, in association with a pharmaceutically acceptable adjuvants, diluents and/or carriers in the manufacture of a medicament for treatment or prophylaxis of pain, chronic inflammatory or neuropathic pain.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substance, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers/diluents include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical composition art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Combinations

In addition to the compounds of the present invention, the pharmaceutical composition of this invention may also contain, or be co-administered (simultaneously or sequentially) with, one or more pharmacological agents of value in treating one or more disease conditions referred to herein.

The pain treatment defined herein may be applied as a sole therapy or may involve, in addition to the compound used according to the invention, administration of other analgesics or adjuvant therapy. Such therapy may for example include, in combination for simultaneous, separate or sequential use one or more of the following categories of pain-relieving ingredients
a) opioid analgesics, for example morphine, ketobemidone or fentanyl
b) analgesics of the NSAID or COX-1 or COX-2 class, for example ibuprofene, naproxene, selecoxib or acetylsalicylic acid, and their analogues containing nitric oxide-donating groups
c) analgesic adjuvants such as amitriptyline, imipramine, duloxetine or mexiletine
d) NMDA antagonists for example ketamine, memantine or dextrometorfan
e) sodium channel blocking agents, for example lidocaine or mexiletine
f) anticonvulsants, for example carbamazepine, topiramate or lamotrigine
g) anticonvulsant/analgesic amino acids such as gabapentin or pregabalin
h) cannabinoids
i) antibodies directed towards NGF or TNF-alpha.
   Biological Tests
   In Vivo Experiments

The compounds used according to the invention when given by systemic administration to mice or rats, specifically reduce pain in the rat carrageenan test as described by Tonussi and Ferreira (Pain 1992, 48, 421-427).

It can therefore be inferred that the compounds can be used as therapeutic agents to relieve pain of various origins.

### Monoarthritis Induced by Carrageenan

One single injection of carrageenan directly into the rat knee joint produces symptoms peaking at 3 to 4 hours and lasting for one to two days, and which are sensitive to treatment by most anti-nociceptive drugs (Tonussi and Ferreira, Pain 1992, 48, 421-427).

Experimental Procedures

Under isoflurane anesthesia, 50 μL of carrageenan (7.5 mg/mL) was injected into the left tibio-tarsal (ankle) joint from the dorsal side. This injection causes a localized inflammation increasing to a maximum between 4 h to 8 h after induction, after which it gradually decreases, and the animals display decreased weight bearing on and guarding of the limb.

Testing Procedure: Paw Print Setup

The Paw Print walkway consists of a 100×10 cm path with an access point at one short end and an exit at the other, and the rats are trained to make a swift, continuous passage. The walkway has a glass floor where light is projected into one long edge via fiber optics. Projecting the light in this fashion allows for virtually complete internal reflection within the glass floor. Only where an object touches the glass, such as the placement of a rat paw, light is scattered at the point of contact and produces an illuminated print (Betts, Duckworth, Eng. Med. 1978, 7, 223-228) that is recorded by a wide-angle camera placed under the walkway. The light intensity (range 0-255) of the illumination depends on the degree of contact against the floor and increases with the applied pressure (Clarke, Physiology & Behavior 1995, 58, 415-419). The Paw Print setup is a modified version of the CatWalk introduced by Hamers, Lankhorst, van Laar, Veldhuis, Gispen (J. Neurotrauma 2001, 18, 187-201), and has a computerized gait detection algorithm extracting several parameters pertaining to the rat's gait pattern and weight-bearing.

The animals were trained to cross the walkway twice, one and two days before induction of monoarthritis. Recordings were subsequently made on the day of testing, immediately before, and three and five hours after carrageenan injection, corresponding to two and four hours after per oral administration of test compound. The following parameters were used for analysis:

Weight-bearing per paw=The total sum of maximum light intensities for all pixels in the print of one paw is multiplied by the number of pixels of the print area. A paw placement consists of a number of pixels with light intensity above a certain threshold value (80 in this study). During the time course of one paw placement, a maximum value of light intensity is recorded for each pixel.

Based on the weight-bearing results, the Paw Print algorithm chooses the median print per paw in one walkway crossing. The median weight-bearing of each paw constitutes a percentage of the sum of all four paws' median weight-bearing results. The difference in percent weight-bearing between the two hind paws is shown (“Walking pain”).

The test compounds were administered 60 min after induction of monoarthritis.

Data Analysis

Values are displayed as mean values ±SEM. Results were analysed using 1-Way ANOVA followed by Newman-Keuls for comparison between treatment groups for data obtained at each time-point after administration. The level of significance was set at p<0.05 (SigmaStat® 2.03).

Results

Paw Print Setup

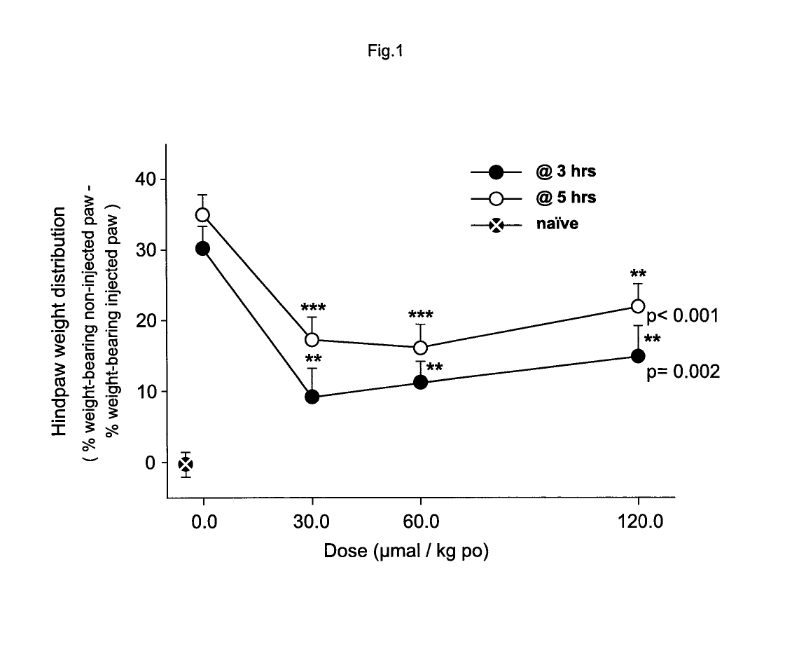

The weight-bearing of each paw before induction of monoarthritis, constituting a percentage of the sum of all paws' median weight-bearing results as given by the Paw Print algorithm, ranged between 21.8±1.6 and 24.6±1.1% (mean ±SEM) for the hind paws respectively. Three and five hours after induction of monoarthritis by carrageenan, the hindpaw weight distribution was 30.2±3.2 and 34.9±2.8% (mean ±SEM), and 22.1±4.6 and 39.0±2.1% (mean ±SEM) at the different test times, compared to values of hindpaw weight distribution of −0.2±1.8 and −2.6±1.0 (mean ±SEM) before carrageenan injection. FIG. 1 shows the effects of 5-Chloro-N²-[(1S)-1-(5-fluoropyridin-2-yl)ethyl]-N⁴-(5-isopropoxy-1H-pyrazol-3-yl)pyrimidine-2,4-diamine on distribution of weight-bearing between the two hind legs in carrageenan-induced monoarthritis in rat, assessed in the Paw Print setup. The median weight-bearing of each paw constitutes a percentage of the sum of all four paws' median weight-bearing results. The difference in percent weight-bearing between the two hind paws are shown. 5-Chloro-N²-[(1S)-1-(5-fluoropyridin-2-yl)ethyl]-N⁴-(5-isopropoxy-1H-pyrazol-3-yl)pyrimidine-2,4-diamine was administered per oral, 1 h after induction of arthritis by carrageenan. (n=8 per group). Statistical analysis was performed by means of 1-way ANOVA followed by the Newman-Keul Method for all pairwise multiple comparisons. The different dose groups treated with 5-Chloro-N²-[(1S)-1-(5-fluoropyridin-2-yl)ethyl]-N⁴-(5-isopropoxy-1H-pyrazol-3-yl)pyrimidine-2,4-diamine were compared statistically to a control group of vehicle treated rats. The level of significance (*) was set at p<0.05.

All three doses of 5-Chloro-N²-[(1S)-1-(5-fluoropyridin-2-yl)ethyl]-N⁴-(5-isopropoxy-1H-pyrazol-3-yl)pyrimidine-2,4-diamine, 30, 60 and 120 μmol/kg, significantly reduced the hindpaw weight distribution compared to the vehicle group, both at two and four hours after per oral administration. In the dose interval tested, no apparent dose dependence was observed. Thus, the dose 30 μmol/kg reduced the difference in weight bearing between the hind paws from 30.2±3.2% at three hours after carrageenan in the vehicle group, to 9.2±4.0%, whereas the dose 120 μmol/kg reduced the difference in weight bearing to 14.9±4.4% (mean ±SEM).

### CONCLUSIONS

The results demonstrate that there was a significant effect by all doses of the TrkA antagonists 5-Chloro-N²-[(1S)-1-(5-fluoropyridin-2-yl)ethyl]-N⁴-(5-isopropoxy-1H-pyrazol-3-yl)pyrimidine-2,4-diamine (30 μmol/kg, 60 μmol/kg and 30 μmol/kg p.o.) on the inflammatory pain induced by intra-articular ankle injection of carrageenan. Carrageenan induced decreased weight bearing measured using the Paw Print computerized gait detection algorithm.

Trk A Assay Format

Trk A kinase activity was measured for its ability to phosphorylate synthetic tyrosine residues within a generic polypeptide substrate using an Amplified Luminescent Proximity Assay (Alphascreen) technology (PerkinElmer, 549 Albany Street, Boston, Mass.).

To measure Trk A kinase activity, the intracellular domain of a HIS-tagged human Trk A kinase (amino acids 442-796 of Trk A, Swiss-Prot Primary Accession Number P04629) was expressed in SF9 cells and purified using standard nickel column chromatography. After incubation of the kinase with a biotinylated substrate and adenosine triphosphate (ATP) for 20 minutes at room temperature, the kinase reaction was stopped by the addition of 30 mM ethylenediaminetetraacetic acid (EDTA). The reaction was performed in 384 well microtitre plates and the reaction products were detected with the addition of strepavidin coated Donor Beads and phosphotyrosine-specific antibodies coated Acceptor Beads using the EnVision Multilabel Plate Reader after an overnight incubation at room temperature.

| | |
|---|---|
| Peptide substrate | PolyEY-biotin (PGT-bio.) |
| ATP Km | 70 μM |
| Assay conditions | 0.838 ng/ml Trk A, 9 mM HEPES, 45 μg/ml BSA, |
| | 10 mM MnCl₂, 5 nM PGT-bio, 0.01% Triton ® |
| | X-100, 70 μM ATP |
| Incubation | 20 minutes, room temperature |
| Termination/ | 6.3 mM HEPES, 30 mM EDTA, 525 μg/ml BSA, |
| Detection | 40 mM NaCl, 0.007% Triton ® X-100, 12 ng/ml |
| conditions | of Donor Beads, 12 ng/ml of Acceptor Beads |
| Detection incubation | overnight, room temperature |
| Fluometer settings | Excitation = 680 nM Emission = 570 nM |
| | Excitation Time = 180 ms Total Measurement |
| | Time = 550 ms |

Although the pharmacological properties of the compound of invention as mentioned above vary with structural change, in general activity possessed by compound of invention as mentioned above may be demonstrated at IC₅₀ concentrations (concentrations to achieve 50% inhibition) or doses in the range of (0.01 μM to 10 μM).

When tested in the above in-vitro assay the Trk inhibitory activity of the following examples was measured at the following IC₅₀s.

| Compound | IC₅₀ (μM) |
|---|---|
| (S)-5-Bromo-N⁴-(5-cyclopropyl-1H-pyrazol- | 0.005 |
| 3-yl)-N²-(1-(5-fluoropyridin-2- | |
| yl)ethyl)pyrimidine-2,4-diamine | |
| (S)-5-Chloro-N⁴-(5-cyclopropyl-1H-pyrazol- | 0.008 |
| 3-yl)-N²-(1-(5-fluoropyridin-2- | |
| yl)ethyl)pyrimidine-2,4-diamine | |
| (S)-5-Fluoro-N⁴-(5-cyclopropyl-1H-pyrazol- | 0.010 |
| 3-yl)-N²-(1-(5-fluoropyridin-2- | |
| yl)ethyl)pyrimidine-2,4-diamine | |

1-15. (canceled)

16. A method of treatment or prophylaxis of pain, in a patient suffering from, or at risk of, said disease, which comprises administering to the patient a therapeutically effective amount of a compound selected from:
(S)-5-bromo-N²-(1-(5-fluoropyridin-2-yl)ethyl)-N⁴-(5-isopropoxy-1H-pyrazol-3-yl)pyrimidine-2,4-diamine;
5-chloro-N²-[(1S)-1-(5-fluoropyridin-2-yl)ethyl]-N⁴-(5-isopropoxy-1H-pyrazol-3-yl)pyrimidine-2,4-diamine;
(S)-5-bromo-N²-(1-(3,5-difluoropyridin-2-yl)ethyl)-N⁴-(5-isopropoxy-1H-pyrazol-3-yl)pyrimidine-2,4-diamine;
(S)-5-chloro-N²-(1-(3,5-difluoropyridin-2-yl)ethyl)-N⁴-(5-isopropoxy-1H-pyrazol-3-yl)pyrimidine-2,4-diamine;
5-fluoro-N²-[(1S)-1-(5-fluoropyridin-2-yl)ethyl]-N⁴-(5-isopropoxy-1H-pyrazol-3-yl)pyrimidine-2,4-diamine;
(S)—N²-(1-(3,5-difluoropyridin-2-yl)ethyl)-5-fluoro-N⁴-(5-isopropoxy-1H-pyrazol-3-yl)pyrimidine-2,4-diamine;
(S)-5-bromo-N⁴-(5-cyclopropyl-1H-pyrazol-3-yl)-N²-(1-(5-fluoropyridin-2-yl)ethyl)pyrimidine-2,4-diamine;
(S)-5-chloro-N⁴-(5-cyclopropyl-1H-pyrazol-3-yl)-N²-(1-(5-fluoropyridin-2-yl)ethyl)pyrimidine-2,4-diamine;
(S)-5-chloro-N⁴-(5-cyclopropyl-1H-pyrazol-3-yl)-N²-(1-(3,5-difluoropyridin-2-yl)ethyl)pyrimidine-2,4-diamine; or
5-chloro-N²-[(1S)-1-(5-fluoropyridin-2-yl)ethyl]-N⁴-(5-methyl-1H-pyrazol-3-yl)pyrimidine-2,4-diamine, or a pharmaceutically acceptable salt thereof, in the manufacture of a medicament for treatment or prophylaxis of pain.

17. The method according to claim 16, wherein said pain is selected from chronic inflammatory pain or neuropathic pain.

18. The method according to claim 16 wherein the compound is comprised in a pharmaceutical composition, in association with a pharmaceutically acceptable adjuvants, diluents and/or carriers.

19. The method according to claim 16 where the pharmaceutically acceptable salt is sulphate or maleate.

20. The method according to claim 16, wherein said use is therapeutic.

21. The method according to claim 16, wherein said use is prophylactic.

22. The method according to claim 16 wherein said pain is caused by chemical, mechanical, radiation, thermal, infectious or inflammatory tissue trauma.

23. The method according to claim 16 wherein said pain is posttraumatic pain, or pain caused by headache and migraine, arthritic and inflammatory conditions selected from pain caused by osteo arthritis and rheumatoid arthritis, myofascial and low back pain associated with chronic inflammation, bone diseases or cancers.

24. The method according to claim 16 wherein said pain is of central or peripheral origin selected from pain caused by trigeminal neuralgia, postherpetic neuralgia, painful diabetic mono/poly neuropathy, and pain associated with nerve damage, spinal cord injury central post stroke, multiple sclerosis or Parkinson's disease.

25. The method according to claim 16 wherein said pain is of visceral origin selected from pain caused by ulcer, dysmenorrhea, endometriosis, IBS and dyspepsia.

26. The method according to claim 16, wherein the daily dose of the compound is in the range of from about 0.1 mg to about 1000 mg.

27. The method according to claim 16, wherein the daily dose of compound is in the range of from about 1 mg to about 750 mg.

28. The method according to claim 16, wherein the daily dose of compound is in the range of from about 1 mg to about 500 mg.
